(19) United States
(12) Patent Application Publication
(10) Pub. No.: US 2023/0174950 A1
(43) Pub. Date: Jun. 8, 2023

(54) Kidney Organoids Having a Nephron-like Structure and Methods of Preparing the Same

(71) Applicants: THE CATHOLIC UNIVERSITY OF KOREA INDUSTRY-ACADEMIC COOPERATION FOUNDATION, Seoul (KR); POSTECH RESEARCH AND BUSINESS DEVELOPMENT FOUNDATION, Pohang-si (KR)

(72) Inventors: Yong Kyun KIM, Seongnam-si (KR); Dong-Woo CHO, Seoul (KR); Sun Ah NAM, Gimpo-si (KR); Jin Won KIM, Seoul (KR); Jae Yeon LEE, Pohang-si (KR); Jae Yun KIM, Pohang-si (KR)

(21) Appl. No.: 17/995,114

(22) PCT Filed: Nov. 24, 2020
(86) PCT No.: PCT/KR2020/016709

§ 371 Date: Sep. 30, 2022

(30) Foreign Application Priority Data
Apr. 1, 2020 (KR) 10-2020-0039625

(51) Int. Cl. C12N 5/071; A61K 35/22

(52) U.S. Cl. CPC C12N 5/0697; A61K 35/22; C12N 5/0686; C12N 2506/45; C12N 2513/00; C12N 2533/54; C12N 2533/90

(57) ABSTRACT

The present invention relates to a kidney organoid having a nephron-like structure and a production method therefor. A kidney organoid culture system using kidney dECM hydrogels according to the present invention induced the vascularization of the kidney organoid and the expression of podocytes, tubular transporters, and cilium genes, and has an effect of forming a more mature nephron-like structure. Therefore, the kidney organoid produced by the method of the present invention is an option for treating nephron loss through transplantation into humans, and is expected to be utilized as a kidney on a chip, which is an in vitro kidney model.

### TECHNICAL FIELD

The present invention relates to a kidney organoid having a nephron-like structure and a production method therefor.

This application claims priority to and the benefit of Korean Patent Application No. 10-2020-0039625 filed in the Korean Intellectual Property Office on Apr. 1, 2020, and all the contents disclosed in the specification and drawings of that application are incorporated in this application.

### BACKGROUND ART

An extracellular matrix (ECM) forms a three-dimensional network of non-cellular, extracellular macromolecular components present in all tissues and organs. ECM-derived materials are often used in tissue regeneration strategies in the field of regenerative medicine. The ECM consists of collagen, enzymes and glycoproteins, and provides a microenvironment for network and cell growth. Cells and the ECM are components capable of interaction within tissues, and cells modify the composition and structure of the ECM in response to physical and biochemical signals transmitted from the ECM.

Therefore, hydrogels derived from a decellularized tissue-specific ECM can provide functions similar to those of a naturally occurring ECM. Decellularized ECM-based hydrogels are one of the key materials used in tissue engineering with the goal of providing structural integrity and biochemical signals.

Meanwhile, with recent advances in the field of stem cells, several different protocols have been established to generate kidney organoids from human pluripotent stem cells (hPSCs). hPSC-derived kidney organoids have segmental structures that include podocytes, proximal tubules and distal tubules as nephron-like arrangements. Comparative analysis of hPSC-kidney organoids in vitro and kidney tissue in vivo demonstrated the fact that kidney organoids recapitulate human kidney development. However, the problem of limited vascularization and immaturity of nephron-like structures still remains a challenge to be overcome.

To overcome the aforementioned problem, previous studies have developed methods for transplantation of kidney organoids into animal kidneys or chick chorioallantoic membranes, or methods for ex vivo transplantation into microfluidic culture systems. Such a challenge contributed to the improvement in vascularization and maturation of nephron-like structures of kidney organoids in vitro, respectively. However, there is a need for a new challenge because vascularization and maturation are still insufficient compared to adult kidneys.

### DISCLOSURE

### Technical Problem

The present inventors confirmed that when a decellularized kidney extracellular matrix is cultured with kidney organoids, it is possible to promote vascularization and maturation of kidney organoids, thereby completing the present invention.

Therefore, an object of the present invention is to provide a method for producing a kidney organoid having a nephron-like structure, the method including culturing a kidney organoid in a collagenous three-dimensional matrix including a decellularized kidney extracellular matrix.

Another object of the present invention is to provide a method for producing a kidney organoid having a nephron-like structure, the method including transplanting a collagenous three-dimensional matrix including a decellularized kidney extracellular matrix; and a kidney organoid cultured in the collagenous three-dimensional matrix into the kidneys of animals other than humans.

Still another object of the present invention is to provide a kidney organoid having a nephron-like structure produced by the method according to the present invention.

Yet another object of the present invention is to provide a collagenous three-dimensional matrix for producing a kidney organoid having a nephron-like structure, including a decellularized kidney extracellular matrix.

However, the technical objects which the present invention intends to achieve are not limited to the technical objects which have been mentioned above, and other technical objects which have not been mentioned will be clearly understood by a person with ordinary skill in the art to which the present invention pertains from the following description.

### Technical Solution

To achieve the aforementioned objects of the present invention, the present invention provides a method for producing a kidney organoid having a nephron-like structure, the method including culturing a kidney organoid in a collagenous three-dimensional matrix including a decellularized kidney extracellular matrix.

Further, the present invention provides a method for producing a kidney organoid having a nephron-like structure, the method including transplanting a collagenous three-dimensional matrix including a decellularized kidney extracellular matrix; and a kidney organoid cultured in the collagenous three-dimensional matrix into the kidneys of animals other than humans.

In an exemplary embodiment of the present invention, the kidney organoid may be derived from a human pluripotent stem cell, but is not limited thereto.

In another exemplary embodiment of the present invention, the decellularized kidney extracellular matrix may be produced from the kidney tissues of animals other than humans, but is not limited thereto.

In still another embodiment of the present invention, the decellularized kidney extracellular matrix may promote the angiogenesis or vascular maturation of the kidney organoid, but is not limited thereto.

In yet another embodiment of the present invention, the decellularized kidney extracellular matrix may increase the expression of one or more genes selected from the group consisting of a tubular epithelial transporter, aquaporin 1 (AQP1), a distal tubule cell marker, E-cadherin, a cilium gene, 3-phosphoinositide-dependent protein kinase-1 (PKD1), a vascular endothelial growth factor (VEGF), a podocyte marker, nephrin (NPHS1), synaptopodin (SYNPO) and a podocyte adult transcription factor (WT1) in a kidney organoid, but is not limited to the above genes.

In yet another exemplary embodiment of the present invention, the collagenous three-dimensional matrix may be a hydrogel, but is not limited thereto.

In yet another exemplary embodiment of the present invention, the transplantation may be performed in the renal subcapsular space of an animal, but is not limited to a specific site in the kidney.

In yet another exemplary embodiment of the present invention, the transplanted kidney organoid may recruit endothelial cells from the kidney of a host animal, but is not limited thereto.

In yet another exemplary embodiment of the present invention, the blood vessels of the transplanted kidney organoid may be connected to the blood vessels of a host animal, but is not limited thereto.

In yet another exemplary embodiment of the present invention, the transplantation may be performed by embedding a kidney organoid in a collagenous three-dimensional matrix, but is not limited thereto.

In addition, the present invention provides a kidney organoid having a nephron-like structure produced by the method according to the present invention.

Furthermore, the present invention provides a collagenous three-dimensional matrix for producing a kidney organoid having a nephron-like structure, including a decellularized kidney extracellular matrix.

### Advantageous Effects

By the present invention, a kidney organoid culture system using kidney dECM hydrogels was used to induce the vascularization for the kidney organoid and the expression of podocytes, tubular transporters, and cilium genes and form a more mature nephron-like structure. Therefore, the kidney organoid produced from human pluripotent stem cells according to the method of the present invention is expected to treat nephron loss by being transplanted to humans or be utilized as a kidney on a chip, which is an in vitro kidney model.

### DESCRIPTION OF DRAWINGS

FIGS. 1A to 1C are views illustrating kidney decellularization and the characteristics of decellularized ECM hydrogels: (FIG. 1A) schematic view illustrating the process of producing a decellularized ECM from porcine kidneys; (FIG. 1B) Hematoxylin-eosin, alcian blue, Masson’s trichrome and anti-fibronectin staining results in a kidney dECM; (FIG. 1C) Results of DNA content analysis of a kidney dECM.

FIGS. 2A to 2F are views illustrating the up-regulation and enhanced vascularization of podocyte and tubular epithelial markers when kidney organoids are cultured in an in vitro kidney dECM: (FIGS. 2A and 2B) Results of observing vasculature marker PCAM1-positive cells and vascular network formation after culture with a kidney dECM for 1 week; (FIGS. 2C to 2F) Real-time quantitative PCR results showing that maturation markers including vascular progenitors, PCAM1, and MCAM and vascular endothelial-cadherin (VE-cadherin) are upregulated when cultured in a kidney dECM.

FIGS. 3A and 3B are views illustrating the enhancement in cell viability and maturity of kidney organoids when cultured in an in vitro kidney dECM: (FIG. 3A) Representative confocal microscopy images of live/dead staining; (FIG. 3B) Representative confocal microscopy images of podocytes and proximal tubular cells.

FIGS. 4A to 4F are views illustrating the vascular network formation of kidney organoids in vivo and maturation of glomerulus-like structures when transplanted with a kidney dECM: (FIG. 4A) Representative images of CD31 immunohistochemical staining in transplanted grafts; (FIG. 4B) Representative confocal microscopy images of MECA32 in transplanted grafts; (FIG. 4C) Representative confocal microscopy images of MECA32 and collagen IV in transplanted grafts; (FIG. 4D) Representative confocal microscopy images comparing the expression of VEGF; (FIG. 4E) Representative microscopic images comparing podocyte structure and alignment with the glomerular basement membrane; (FIG. 4F) Microscopic images showing that podocytes and endothelial cells are aligned to the glomerular basement membrane.

### MODES OF THE INVENTION

Human pluripotent stem cell (hPSC)-derived kidney organoids have segmental structures including nephron-like arrangements of podocytes, and proximal and distal tubules. However, the limited vascularization of nephron-like structures and the resulting immaturity of blood vessels still remain a challenge to be overcome.

An extracellular matrix (ECM) provides the mechanical support and biochemical microenvironment for cell growth and differentiation. The present inventors developed a culture system for hPSC-derived kidney organoids including kidney decellularized extracellular matrix (dECM) hydrogels, and confirmed that the culture system can induce the up-regulation of gene expression for maturation of podocytes and tubular epithelial cells to enhance the angiogenesis of kidney organoids, thereby completing the present invention.

Therefore, the present invention may provide a method for producing a kidney organoid having a nephron-like structure, the method including culturing a kidney organoid in a collagenous three-dimensional matrix including a decellularized kidney extracellular matrix.

As another aspect of the present invention, the present invention may provide a method for producing a kidney organoid having a nephron-like structure, the method including transplanting a collagenous three-dimensional matrix including a decellularized kidney extracellular matrix; and a kidney organoid cultured in the collagenous three-dimensional matrix into the kidneys of animals other than humans.

As used herein, the term “decellularization” refers to the removal of other cellular components, for example, nuclei, cell membranes, nucleic acids, and the like, except for the extracellular matrix from cells or tissues. The term “decellularized extracellular matrix” refers to an extracellular matrix remaining after cellular components such as nuclei, cell membranes, and nucleic acids have been removed from tissues or cells.

As used herein, the term “organoid” refers to an organ-specific cell aggregate made by aggregating and recombining cells isolated from stem cells or organ-derived cells by a three-dimensional culture method, and is an organ analogue of an organ-specific cell type that self-organizes (or self-patterns) through cellular classification and spatially limited lineage commitment in a manner analogous to the in vivo state. Thus, organoids exhibit the native physiology of cells, and have an anatomical structure that mimics the native state of a cell mixture (including not only limited cell types, but also residual stem cells, and proximal physiological niches). Stem cells may be isolated from tissue or organoid fragments. Cells, in which organoids are produced, differentiate in vivo to form organ-like tissues that exhibit multiple cell types that are self-organized to form structures very similar to those of organs. Therefore, the organoid is an excellent model for studying human organs and human organ development in a system that is very similar to in vivo development.

As used herein, the term “nephron” plays a central role in urine production as a basic unit that constitutes the structure and function of the kidneys, and is also referred to as a renal unit. The nephron consists of the renal corpuscle (glomerulus and glomerular capsule), a proximal tubule (proximal convoluted tubule), Henle’s loop, a distal tubule (distal convoluted tubule), and a collecting duct. Normally, 1,000,000 to 1,500,000 nephrons are present in one kidney. Kidney failure occurs when nephron function is paralyzed by an infectious disease such as nephritis, or when the number of nephrons decreases due to other diseases.

In an exemplary embodiment of the present invention, kidney organoids derived from human induced pluripotent stem cells (iPSCs) were inserted into a kidney dECM and cultured in vitro (see Example 2). In another exemplary embodiment of the present invention, kidney organoids having a dECM were transplanted into mouse kidneys, and their vascularization and maturation were concentrated (see Examples 3 and 4).

As a result, in a kidney organoid cultured with a kidney dECM, the expression of a tubular epithelial transporter, aquaporin 1 (AQP1), a distal tubule cell marker, E-cadherin, a cilium gene, 3-phosphoinositide-dependent protein kinase-1 (PKD1), a vascular endothelial growth factor (VEGF), a podocyte marker, nephrin (NPHS1), synaptopodin (SYNPO) and a podocyte adult transcription factor (WT1) was up-regulated, and accordingly, it was confirmed that the nephron structure of the kidneys matured because vascularization was remarkably promoted.

In the present invention, the kidney organoid may be produced by differentiation from human pluripotent stem cells, but is not limited thereto.

As used herein, the term “stem cell” is a cell capable of differentiating into various cells that make up a biological tissue, and refers to undifferentiated cells capable of being regenerated unlimitedly to form specialized cells of tissues and organs. Stem cells are totipotent or multipotent cells which can be developed, and can divide into two daughter stem cells, or one daughter stem cell and one derived (transit) cell, and then proliferate into cells in a mature and intact form of tissue.

As used herein, the term “pluripotent stem cells” refers to stem cells that are completely capable of differentiating into cells constituting the endoderm, mesenchyme, and ectoderm as cells in a state in which cells are developed more than a fertilized egg. According to a specific exemplary embodiment of the present invention, the pluripotent stem cells used in the present invention are embryonic stem cells, embryonic germ cells, embryonic carcinoma cells or induced pluripotent stem cells, and more specifically embryonic stem cells or induced pluripotent stem cells (iPSCs).

In the present invention, the decellularized kidney extracellular matrix may be produced from kidney tissue of animals other than humans. In an exemplary embodiment of the present invention, kidney tissue obtained from a pig was used, but is not limited thereto.

The decellularized kidney extracellular matrix may be produced by a method including the following steps, but is not limited thereto.
(a) cutting the kidney tissue of an animal other than humans into sections having a thickness of 0.01 to 1 mm;
(b) treating the kidney tissue with Triton X-100 dissolved in sodium chloride (NaCl) for 10 to 24 hours;
(c) treating the kidney tissue with DNase for 2 to 10 hours;
(d) sterilizing the kidney tissue; and
(e) lyophilizing the kidney tissue.

In the present invention, the decellularized kidney extracellular matrix may promote the angiogenesis or vascular maturation of the kidney organoid. In an exemplary embodiment of the present invention, it was confirmed that the vascularization of the kidney organoid is promoted and the nephron structure of the kidneys is matured by the decellularized kidney extracellular matrix.

In the present invention, the collagenous three-dimensional matrix may be a hydrogel, but is not limited thereto.

As used herein, the term “hydrogel” may be used interchangeably with the term “hydrated gel,” is a hydrophilic polymer network forming a three-dimensional cross-linkage, and exhibits a protein composition almost similar to native tissue due to its high moisture content. In addition, since the hydrogel is not dissolved in an aqueous environment and is made from various polymers, it has various chemical compositions and physical properties. Furthermore, the hydrogel is easily processed, and thus, may be transformed into various shapes depending on the application. The hydrogel has high biocompatibility due to its high water content and physicochemical similarity to the extracellular matrix.

The decellularized extracellular matrix hydrogel of the kidney according to the present invention may include an extracellular matrix protein including collagen-IV, laminin, heparan sulfate proteoglycan and isoforms thereof.

In the present invention, the transplantation may be performed in the renal subcapsular space of an animal, but is not limited to a specific site in the kidney.

In the present invention, the transplanted kidney organoid may recruit endothelial cells from the kidney of a host animal. Further, the blood vessels of the transplanted kidney organoid may be connected to the blood vessels of a host animal.

In addition, in the present invention, the transplantation may be performed by embedding a kidney organoid in a collagenous three-dimensional matrix, and the kidney organoid embedded in the collagenous three-dimensional matrix may include at least one or more, for example, 5 or more and 30 or less, kidney organoids (or cell aggregates), but is not limited thereto.

As another aspect of the present invention, the present invention may provide a kidney organoid having a nephron-like structure produced by the method according to the present invention.

As still another aspect of the present invention, the present invention may provide a collagenous three-dimensional matrix for producing a kidney organoid having a nephron-like structure, including a decellularized kidney extracellular matrix.

Terms or words used in the specification and the claims should not be interpreted as being limited to typical or dictionary meanings and should be interpreted with a meaning and a concept that are consistent with the technical spirit of the present invention based on the principle that an inventor can appropriately define a concept of a term in order to describe his/her own invention in the best way.

Hereinafter, preferred examples for helping with understanding of the present invention will be suggested. However, the following examples are provided only so that the present invention may be more easily understood, and the content of the present invention is not limited by the following examples.

### EXAMPLES

### Experimental Example. Experimental Materials and Methods

### 1. Decellularization of Kidney Tissue and Production of Decellularized Extracellular Matrix (dECM) Hydrogel

### 1.1. Decellularization of Kidney Tissue

Kidney tissue obtained from a pig was sliced into slices having a thickness of 0.1 to 0.3 mm and washed three times with distilled water for 30 minutes. Next, the slices were treated with 0.5% Triton X-100 (Sigma-Aldrich, USA) in 1 M NaCl (Samchun Chemical Co., Ltd., Korea) for 16 hours. Thereafter, the slices were again washed three times for 1 hour. Remaining cellular components were removed by treatment with DNase at 37° C. for 6 to 7 hours. Subsequently, the tissue slices treated with DNase were washed with phosphate-buffered saline (PBS) for 12 hours, then sterilized with a 0.1% peracetic acid solution for 1 hour, and washed again using distilled water. A decellularized tissue was lyophilized at -80° C., and then used for biochemical characterization and production of kidney dECM hydrogels.

### 1.2. Production of Kidney dECM Hydrogel

A kidney dECM hydrogel was produced by dissolving the previously decellularized kidney tissue in an acetic acid solution. The acetic acid solution included decellularized kidney tissue and pepsin at a mass ratio of 10:1 and was stirred for 72 to 96 hours depending on the concentration of decellularized tissue in the solution. After the dissolution was completed, the acetic acid solution was neutralized using sodium hydroxide and diluted using distilled water to finally make a kidney dECM hydrogel at a required concentration.

### 2. Biochemical Characterization of Kidney dECM

To quantify double-stranded DNA (dsDNA), a kidney dECM was digested at 60° C. for 16 hours using 1 ml of a papain solution (125 µg/ml papain in 0.1 M sodium phosphate containing 5 mM Na2-EDTA and 5 mM cysteine-HCl at a pH of 6.5). Then, dsDNA was isolated from the digested sample using a GeneJET genomic DNA purification kit (Thermo Scientific, USA). 1 µl of the digested sample was loaded into a NanoDrop (Thermo Scientific) and the amount of its contents was determined.

For immunohistochemical analysis, native kidney and decellularized tissues were fixed in 10% formalin, embedded in paraffin, and then a section was made using a microtome. Sectioned samples were stained with hematoxylin and eosin (H&E), alcian blue, Masson’s trichrome and anti-fibronectin. Subsequently, the stained samples were observed under an optical microscope.

### 3. Differentiation of Kidney Organoid

A CMC11 iPSC cell line was obtained from The Catholic University of Korea (male donor). The differentiation of kidney organoids was performed according to a conventional method using cells with a passage number between 30 and 60 (Freedman et al., 2015). Briefly, hPSCs were plated at a density of 5,000 cells/well along with an mTeSR1 medium (Stem Cell Technologies, USA) containing 10 µM Y27632(LC Laboratories, USA) in a 24-well glass plate (LabTek, Australia) coated with 3% GelTrex™ (Thermo Fisher Scientific, USA) (day -3).

The medium was exchanged with mTeSR1 including 1.5% GelTrex on day -2, with mTeSR1 on day -1, with RPMI (Thermo Fisher Scientific) containing 12 µM CHIR99021 (Tocris, UK) on day 0, and with RPMI (Thermo Fisher Scientific) containing a B27 supplement on day 1.5, respectively. Thereafter, a RPMI (Thermo Fisher Scientific) medium containing a B27 supplement was supplied every 2 and 3 days to promote the differentiation of the kidney organoid.

On day 18, the organoid attached to the 24-well plate were microdissected using a 23-gauge injection needle under an inverted phase-contrast microscope. Then, the acquired kidney organoid was placed on an 8-well chamber slide (ibidi, Germany) coated with 0.1% kidney dECM, and RPMI containing a B27 supplement was supplied every 2 and 3 days. On day 25, the kidney organoid was fixed.

### 4. Immunofluorescence and Immunohistochemical Analysis

For immunofluorescence analysis, the organoid was fixed on day 18 unless otherwise stated. For fixation, equal volumes of PBS (Thermo Fisher Scientific) and 8% paraformaldehyde (Electron Microscopy Sciences, USA) were added to the medium for 15 minutes, and then the samples were washed three times with PBS. Fixed organoid cultures were blocked with 5% donkey serum (Millipore, USA) containing 0.3% Triton-X-100/PBS, cultured with a primary antibody in PBS containing 3% bovine serum albumin (Sigma-Aldrich) overnight, and then washed. Thereafter, the organoid cultures were treated with an AlexaFluor secondary antibody (Invitrogen), cultured, washed, stained with DAPI, or mounted using Vectashield H-1000.

After embedding, for single immunohistochemical (IHC) staining, kidneys and kidney organoids were fixed, then embedded in wax and cut transversely into a thickness of 4 µm using a microtome. Some kidney sections and kidney organoid sections were processed and stained with an H&E stain or Masson’s trichrome stain. The other sections were treated for immunohistochemical analysis after embedding. These tissue sections were hydrated with graded ethanol and rinsed with tap water. After dewaxing, the sections were microwave-incubated with a retrieval solution for 10 minutes. The sections were washed with tap water and incubated with methanolic H₂O₂ for 30 minutes for endogenous peroxidase blocking. Next, the sections were cultured with a 0.5% Triton X-100/PBS solution for 15 minutes and rinsed with PBS. Non-specific binding sites were blocked with normal donkey serum (diluted 1:10 in PBS) for 1 hour followed by overnight incubation with a primary antibody at 4° C. The next day, after being rinsed with PBS, the sections were incubated in peroxidase-conjugated donkey anti-mouse or anti-rabbit immunoglobulin G (IgG; Jackson ImmunoResearchLab, USA) for 2 hours, and then washed again with a 0.05 M Tris buffer (pH 7). For detection, the sections were treated with 0.05% 3,3′-diaminobenzidine (DAB) and 0.01% H₂O₂. Thereafter, the sections were washed with distilled water, dehydrated with ethanol and xylene, and then mounted on Canada balsam and observed under an optical microscope.

After embedding, for multiplex immunohistochemical (IHC) staining, tissue and organoid sections were stained with DAB and then treated with methanolic H₂O₂ for 30 minutes to remove the peroxidase remaining from the first staining. Subsequently, the sections were incubated with other primary antibodies. After washing once with PBS, the sections were cultured with peroxidase-conjugated donkey anti-rabbit IgG (Jackson Immuno Research Lab) for 2 hours. For the detection of peroxidase, Vector SG (Vector Laboratories, USA) was used as a chromogen to produce a gray-blue color, which is easily distinguished from a brown stain produced by DAB. The sections were washed with distilled water, dehydrated with graded ethanol and xylene, then mounted on Canada balsam and observed under an optical microscope. The following antibodies were used as primary antibodies: anti-LTL (Vector Labs FL-1321, 1:500 dilution), anti-ZO-1 (Invitrogen 339100, 1:100), anti-NPHS1 (R&D AF4269, 1:500), anti-ECAD (Abcam, ab11512, 1:100), anti-THP (MP Bio, 55140; 1:200), anti-Claudin 1 (Abcam an15098, 1:100), anti-WT1 (Abcam ab89901, 1:100), anti-CD31 (R&D Systems AF3628, 1:200), anti-laminin (Sigma-Aldrich L9393. 1:200), anti-human nuclear antibody (HNA) (Merck Millipore MAB1281, 1:100) and anti-WT1 (Santa Cruz sc-192, 1:100).

### 5. Electron Microscopy Analysis

Adult mouse kidney block samples, transplanted kidney organoids and in vitro kidney organoid samples were fixed at 4° C. overnight using 4% paraformaldehyde and 2.5% glutaraldehyde in a 0.1 M phosphate buffer. After washing with a 0.1 M phosphate buffer, the samples were post-fixed with 1% osmium tetroxide in the same buffer at 4° C. for 1 hour. Subsequently, the samples were dehydrated with a series of graded ethyl alcohol solutions, exchanged through acetone, and then embedded in Epon 812. Thereafter, an ultrathin section (70 to 80 nm) was obtained by an ultramicrotome (Leica Ultracut UCT, Germany). The ultrathin section was double-stained with uranyl acetate and lead citrate, and then observed with a transmission electron microscope (JEM 1010, Japan) at 60 kV. For quantitative analysis, 20 low-magnification (x6,000) fields were randomly selected from each section of cortex, and the number of autophagosomes per 100 µm² was determined.

### 6. Transplantation of Human iPSC-Derived Kidney Organoids

Adherent organoids were microdissected from 24-well plates using a 23-gauge injection needle on day 18 of differentiation, and then carefully transferred to an Eppendorf tube containing RB using a transfer pipette. Harvested kidney organoids were transplanted with 0.1% kidney dECM into the renal subcapsular space of 8-week-old immunodeficient male NOD/SCID mice (Jackson Laboratories, USA).

Briefly, the mice were anesthetized with Zoletil and then the kidneys were exposed through a lateral incision in the back. After about a 2 mm incision in the host kidney capsule with a 23-gauge injection needle, a PE50 tube containing 10 to 20 kidney organoids was carefully placed under the kidney capsule. The kidney organoids and 0.1% kidney dECM were delivered by carefully blowing through the other side of the PE50 tube. Mice were sacrificed 14 days after transplantation (n=3 per group).

### 7. Real-Time Quantitative Polymerase Chain Reaction (RT-qPCR) Analysis

Kidney organoid samples were collected and total RNA was isolated from each sample using an RNAiso plus kit (TAKARA, Japan) according to the manufacturer’s instructions. Complementary DNA was synthesized using a Maxima First Strand cDNA synthesis kit for RT-qPCR (Thermo Fisher Scientific). Gene expression was analyzed with a Power SYBR Green PCR master mix (Applied Biosystems, USA) using a real-time polymerase chain reaction (Applied Biosystems, USA).

### 8. Statistical Analysis

For all quantitative measurements, the entire population was used for statistical significance calculations, and a mean with an n value of 3 was used to calculate standard errors and graphical confidence intervals. Data was then analyzed using the Mann-Whitney test or the Kruskal-Wallis test to determine the significance between groups. Error bars in each graph represent -2 SEM (standard error of the mean) with a 95% confidence interval. A single asterisk was used for a p-value of <0.05, two asterisks for p <0.01, and three asterisks for p <0.001.

### Example 1. Decellularization of Kidneys and Characteristics Of Decellularized Kidney ECM Hydrogel

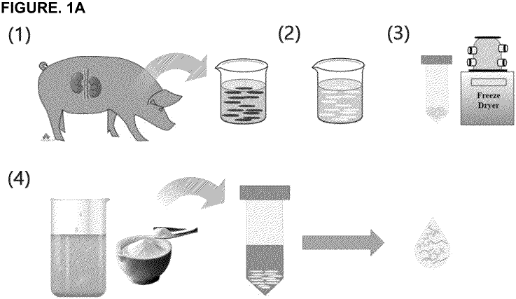

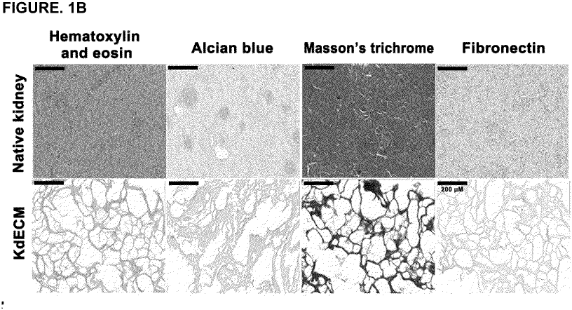

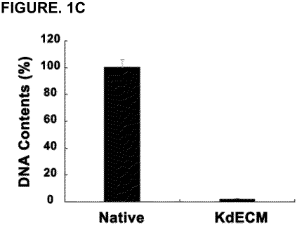

Kidneys were decellularized as illustrated in the schematic view of FIG. 1A.

First, it was confirmed that there was no visible cellular component in the kidney dECM through H&E staining. The remaining fibronectin and collagen components were visually evaluated by alcian blue, anti-fibronectin and Masson’s trichrome staining, respectively. As a result, as illustrated in FIG. 1B, it was confirmed that fibronectin and collagen, which are major ECM components of the kidneys, were well preserved in the decellularized kidney tissue.

Further, the cellular components remaining after decellularization were evaluated. As a result, as illustrated in FIG. 1C, the DNA content of kidney dECM remained at a level of 2.29% compared to that of native kidney tissue, and only 0.64 ng of DNA/mg remained. This indicates that most cellular components were successfully removed.

### Example 2. Culture of Kidney Organoid on in Vitro Kidney dECM and Its Effect

To confirm the effect of a kidney dECM in the culture of kidney organoids, the present inventors generated kidney organoids from human iPSCs using an adherent culture differentiation protocol and then purified the kidney organoids by microdissection from the peripheral stroma. The kidney organoids obtained as described above were inserted into a kidney dECM and cultured.

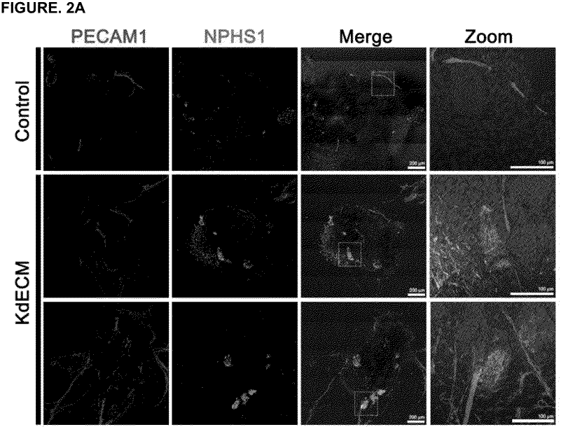

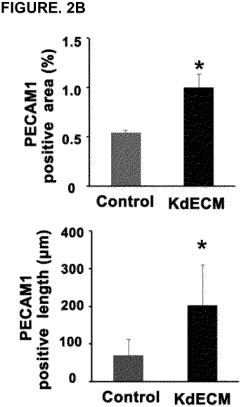

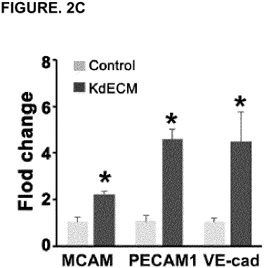

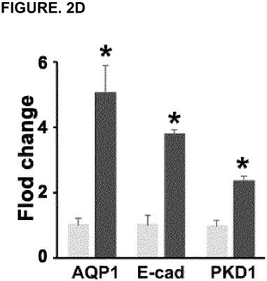

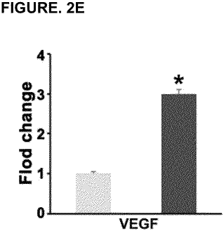

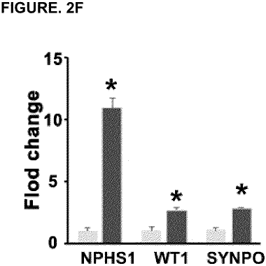

As a result, as illustrated in FIGS. 2A and 2B, the kidney dECM increased vasculature marker PCAM1-positive cells and vascular network formation in kidney organoids within 1 week. The vascular network appeared to extensively surround the nephron-like structure. In addition, it was confirmed that the area, length and diameter of PCAM1-positive vasculature increased in kidney organoids inserted into the kidney dECM compared to the control (FIG. 2B).

Furthermore, it was confirmed through a real-time quantitative polymerase chain reaction (RT-qPCR) that vascular progenitor and maturation markers, including PCAM1 and MCAM, as well as vascular endothelial cadherin (VE-cadherin), were increased when the kidney organoids were cultured in the kidney dECM (FIG. 2C).

When the kidney organoids were transplanted into mouse kidneys, cultured in a microfluidic system, or transplanted into chick chorioallantoic membranes, enhanced vascularization of kidney organoids as well as progressive morphogenesis of tubular structures can be observed. The present inventors determined an enhanced vascularization effect according to the culture with the kidney dECM in regard to the maturation of tubular epithelial cells. As a result, as illustrated in FIG. 2D, it was confirmed that when the kidney organoids were cultured on the kidney ECM, the expression of a tubular epithelial transporter, aquaporin 1 (AQP1), a distal tubule cell marker, E-cadherin, a cilium gene, and 3-phosphoinositide-dependent protein kinase-1 (PKD1) was up-regulated.

In consideration of the fact that glomerular vascularization is essential for human podocyte development, the present inventors also investigated the effect of a kidney dECM on the vascularization of the glomerular compartment. As a result, in the kidney organoids inserted into the kidney dECM, the PCAM1-positive vasculature partially penetrated into NPHS1-positive cells, whereas this phenomenon was not observed in the control (FIG. 2A).

In the development of the kidneys, at the s-shape body stage, vascular endothelial growth factor A (VEGF-A) produced by podocyte progenitors contributes to subsequent podocyte maturation by attracting infiltrating endothelial cells. Thus, the present inventors analyzed the gene expression of VEGF-A and podocytes. As a result, it was confirmed that VEGF was up-regulated in organoids cultured with the kidney dECM (FIG. 2E).

When the kidney organoids were cultured in a kidney dECM, a podocyte marker, nephrin (NPHS 1), synaptopodin (SYNPO) and a podocyte adult transcription factor (WT1) were up-regulated (FIG. 2F). When taken together, the above results indicate that the kidney dECM up-regulates VEGF expression and induces infiltrating glomerulus-like structures by a PCAM1-positive vasculature accompanied by podocyte maturation.

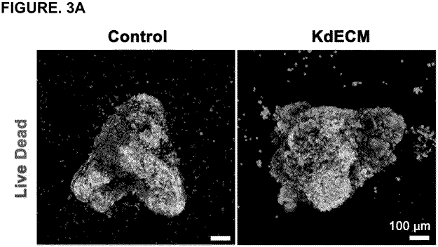

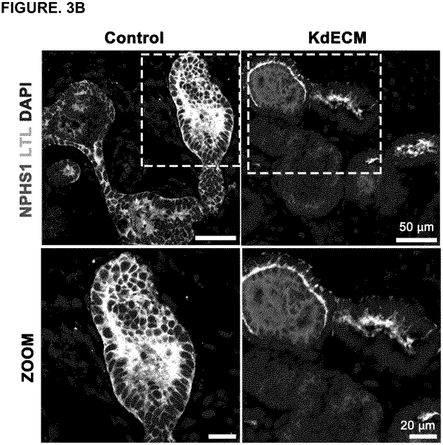

In addition, as illustrated in FIGS. 3A and 3B, it was confirmed that when the kidney organoids were cultured in a kidney dECM, the survival of cells constituting kidney organoids was enhanced (FIG. 3A) and the polarity of proximal tubular epithelial cells was enhanced.

### Example 3. In Vivo Transplantation of Kidney Organoids Including kidney dECM and Its Effect

Transplantation of human kidney organoids into mouse kidneys was known to enhance the formation of a perfusable vasculature that facilitated the maturation of glomerulus-like and tube-like structures in kidney organoids. Thus, considering that a kidney dECM up-regulates VEGF expression and enhances the vascularization of kidney organoids, the present inventors hypothesized that transplanting kidney organoids having a kidney dECM could accelerate vascularization in the transplanted graft, through which more advanced morphogenesis could be elicited in nephron-like structures of kidney organoids.

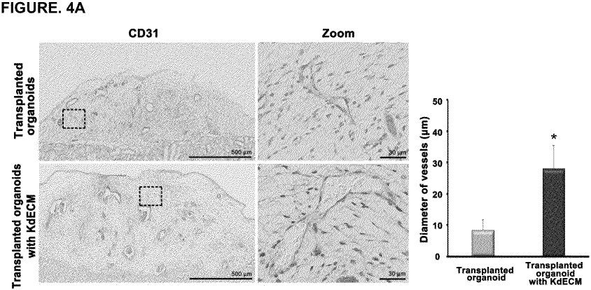

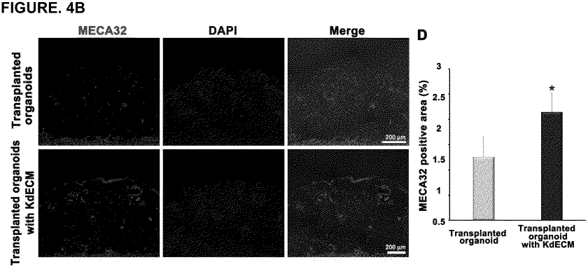

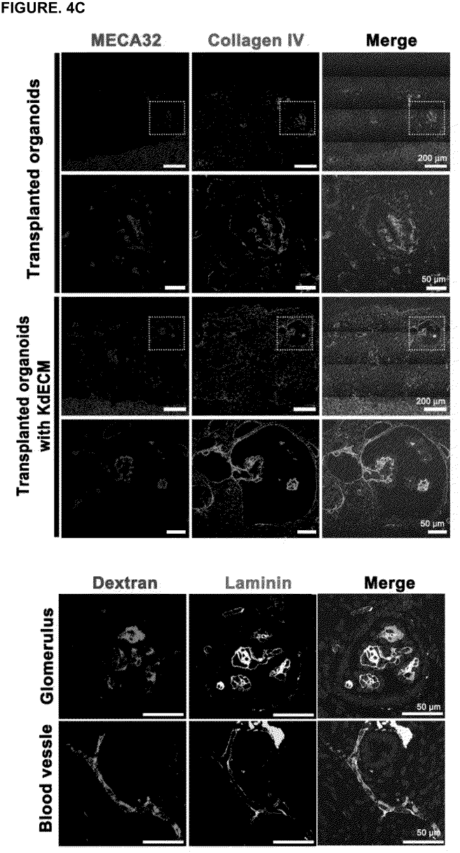

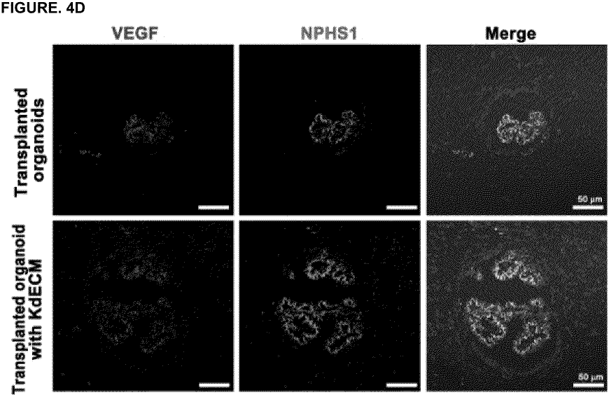

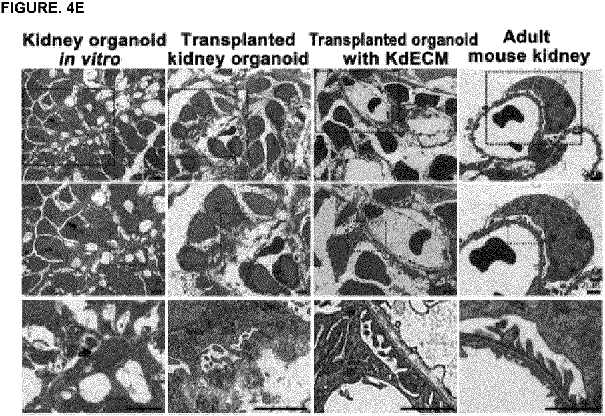

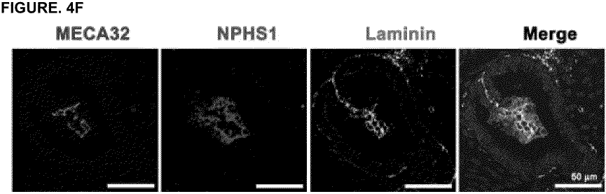

To test the above hypothesis, the present inventors transplanted kidney organoids derived from human iPSCs having a kidney dECM under the kidney capsule of immunodeficient NOD-SCID mice for engraftment. Blood vessels with CD31-positive cells were abundantly formed in transplanted grafts for 2 weeks after transplantation (FIG. 4A). It was found that the vessel diameter of grafts transplanted with the kidney dECM was larger than that of grafts lacking a kidney dECM and transplanted (FIG. 4A).

Furthermore, mouse endothelial cells (MECA32+) were abundantly observed within transplanted kidney organoid grafts and glomerulus-like structures (FIG. 4B). More abundant MECA32-positive cells were observed in the transplanted kidney organoids having the kidney dECM compared to the control, suggesting that the kidney dECM has an effect of recruiting endothelial cells from the mouse kidneys to transplanted grafts.

Collagen IV, a major component of the basement membrane, is essential for vascular integrity, stability and functionality during development. Thus, the present inventors investigated the expression of collagen in transplanted kidney organoids, considering that collagen IV is the most abundant protein in a kidney dECM. Confocal fluorescence microscopy revealed that transplanted kidney organoids having a kidney dECM had greater expression of collagen IV in glomerular capillaries and peritubular capillaries compared to kidney dECM-free transplanted kidney organoids (top of FIG. 4C).

Further, to confirm the integrity of the vasculature formed in kidney organoids transplanted with a kidney dECM, mice were injected with dextran labeled with 500 kDa fluorescein isothiocyanate (FITC) into the tail vein. FITC-labeled dextran was present inside the blood vessels and capillaries of the glomerulus-like structure in the transplanted kidney organoids (bottom of FIG. 4C), suggesting that the vasculature of the transplanted kidney organoids is connected to the infiltrating renal vasculature derived from the host mouse to maintain vascular integrity.

When the above results are taken together, the kidney dECM accelerated the recruitment of endothelial cells from the host mouse kidney, confirming that by increasing collagen IV in the basement membrane, a vascular network is formed and the integrity of blood vessels is maintained.

### Example 4. In Vivo Transplantation of Kidney Organoids Including kidney dECM and Maturation Effect of Glomerulus-Like Structures

Podocytes are cells in the outer layer of the kidney glomerular capillary loop. As the first step in forming urine, the glomeruli filter the blood to send back large molecules such as proteins and allow small molecules such as water, salts and sugars to pass through. Long projections or foot processes of podocytes wrap around capillaries and rest on the basement membrane of the glomerulus. The foot processes are connected by a porous structure called the slit diaphragm.

In vitro studies showed that the up-regulation of VEGF expression appeared in addition to increased glomerular vascularization and podocyte maturation during culture with a kidney dECM (FIGS. 2A to 2F). Thus, the present inventors investigated the degree of maturation of glomerulus-like structures during transplantation with a kidney dECM in consideration of the enhanced vascularization after transplantation of kidney organoids.

As a result of observation by confocal fluorescence microscopy, it was confirmed that the expression of VEGF was increased more in the glomerulus-like structures of transplanted kidney organoids having a kidney dECM compared to kidney dECM-free transplanted kidney organoids (FIG. 4D).

In addition, in order to determine the ultrastructure of cells, an additional structural analysis using transmission electron microscopy (TEM) was performed, and in vitro kidney organoids, kidney organoids transplanted in vivo and adult mouse kidneys were compared with one another.

As a result, as illustrated in FIG. 4E, in in vitro kidney organoids, podocytes had an immature structure with apical microvilli and intermittently arranged along glomerular basement membrane (GBM)-like tracks. In comparison, in the case of kidney organoids transplanted alone, erythrocyte fragments were observed in the transplanted kidney organoids, indicating that capillaries can be formed (FIG. 4E). However, transplanted kidney organoids lacked a bona fide foot process with well-organized tertiary interdigitation along the GBM. Furthermore, the Bowman’s capsule of the transplanted organoid was structurally similar to the Bowman’s capsule of the adult mouse, but had a substantially thicker capsule layer than the Bowman’s capsule of the adult mouse. In contrast, the podocytes of kidney organoids transplanted with a kidney dECM had secondary or tertiary foot processes that engaged the GBM similar to those of the adult mouse kidney (FIG. 4E). Further, when kidney organoids were transplanted with a kidney dECM, the GBM was well-organized and aligned with podocytes and endothelial cells compared to the adult kidneys of a mammal (FIG. 4F). The aforementioned results demonstrate the fact that the kidney dECM contributes to the maturation of glomerulus-like structures in transplanted kidney organoids.

In summary, when kidney organoids having a kidney dECM were transplanted under the kidney capsule in immunodeficient mice, the recruitment of endothelial cells from the kidney of the host mouse was promoted and the integrity of blood vessels was maintained. In addition, in transplanted kidney organoids having a kidney dECM, slit diaphragm-like structures were more organized compared to kidney dECM-free slit diaphragm-like structures.

These results suggest the fact that a microenvironment provided from a kidney dECM hydrogel promotes angiogenesis and maturation of iPSC-derived kidney organoids, and it is expected that the microenvironment generates a kidney with blood vessels on a chip or can be applied to regenerative therapy.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

The present invention induced the vascularization of the kidney organoid, induced the expression of podocytes, tubular transporters and cilium genes and formed a more mature nephron-like structure using a kidney organoid culture system using kidney dECM hydrogels. Therefore, the kidney organoid produced from human pluripotent stem cells according to the production method of the present invention is expected to treat nephron loss by being transplanted to humans or be utilized as a kidney on a chip, which is an in vitro kidney model, and thus is expected to have great industrial utility value.

1. A method for producing a kidney organoid having a nephron-like structure, the method comprising culturing a kidney organoid in a collagenous three-dimensional matrix comprising a decellularized kidney extracellular matrix.

2. A method for producing a kidney organoid having a nephron-like structure, the method comprising transplanting a collagenous three-dimensional matrix comprising a decellularized kidney extracellular matrix; and a kidney organoid cultured in the collagenous three-dimensional matrix into the kidneys of animals other than humans.

3. The method of claim 2, wherein the kidney organoid is derived from pluripotent stem cells.

4. The method of claim 2, wherein the decellularized kidney extracellular matrix is produced from a kidney tissue of an animal other than humans.

5. The method of claim 2, wherein the decellularized kidney extracellular matrix promotes the angiogenesis or vascular maturation of the kidney organoid.

6. The method of claim 2, wherein the decellularized kidney extracellular matrix increases the expression of one or more genes selected from the group consisting of a tubular epithelial transporter, aquaporin 1 (AQP1), a distal tubule cell marker, E-cadherin, a cilium gene, 3-phosphoinositide-dependent protein kinase-1 (PKD1), a vascular endothelial growth factor (VEGF), a podocyte marker, nephrin (NPHS1), synaptopodin (SYNPO) and a podocyte adult transcription factor (WT1) in a kidney organoid.

7. The method of claim 2, wherein the collagenous three-dimensional matrix is a hydrogel.

8. The method of claim 2, wherein the transplantation is performed in the renal subcapsular space of an animal.

9. The method of claim 2, wherein the transplanted kidney organoid recruits endothelial cells from the kidney of a host animal.

10. The method of claim 2, wherein the blood vessels of the transplanted kidney organoid is connected to the blood vessels of a host animal.

11. The method of claim 2, wherein the transplantation is performed by embedding a kidney organoid in a collagenous three-dimensional matrix.

12. (canceled)

13. A collagenous three-dimensional matrix for producing a kidney organoid having a nephron-like structure, comprising a decellularized kidney extracellular matrix.

14. The method of claim 1, wherein the kidney organoid is derived from pluripotent stem cells.

15. The method of claim 1, wherein the decellularized kidney extracellular matrix is produced from a kidney tissue of an animal other than humans.

16. The method of claim 1, wherein the decellularized kidney extracellular matrix promotes the angiogenesis or vascular maturation of the kidney organoid.

17. The method of claim 1, wherein the decellularized kidney extracellular matrix increases the expression of one or more genes selected from the group consisting of a tubular epithelial transporter, aquaporin 1 (AQP1), a distal tubule cell marker, E-cadherin, a cilium gene, 3-phosphoinositide-dependent protein kinase-1 (PKD1), a vascular endothelial growth factor (VEGF), a podocyte marker, nephrin (NPHS1), synaptopodin (SYNPO) and a podocyte adult transcription factor (WT1) in a kidney organoid.

18. The method of claim 1, wherein the collagenous three-dimensional matrix is a hydrogel.
